(19) United States
(12) Patent Application Publication
(10) Pub. No.: US 2025/0321345 A1
(43) Pub. Date: Oct. 16, 2025

(54) DEVIATION ACQUISITION METHOD FOR SINGLE-PHOTON EMISSION COMPUTED TOMOGRAPHY SYSTEM AND COMPUTER DEVICE

(71) Applicant: Shanghai United Imaging Healthcare Co., Ltd., Shanghai (CN)

(72) Inventors: Songsong Tang, Shanghai (CN); Beien Wang, Shanghai (CN); Yilin Liu, Shanghai (CN); Yun Dong, Shanghai (CN)

(21) Appl. No.: 19/174,970

(22) Filed: Apr. 10, 2025

(30) Foreign Application Priority Data
Apr. 10, 2024 (CN) 202410432660.0

(51) Int. Cl. G01T 7/00; G01T 1/164

(52) U.S. Cl. CPC G01T 7/005; G01T 1/1642

(57) ABSTRACT

The present disclosure relates to a deviation acquisition method for a single-photon emission computed tomography system, which includes: controlling a target object to move to a first target position through a translation stage, and determining an actual detection position of the target object detected by a system detector in a current pose; obtaining at least one first expected pose deviation; determining at least one expected detection position of the target object based on the first target position and the at least one first expected pose deviation, and obtaining at least one first position difference between the at least one expected detection position and the actual detection position; and determining an actual pose deviation between the expected pose and the current pose of the system detector based on the at least one first position difference corresponding to the at least one first expected pose deviation.

### CROSS-REFERENCE TO RELATED APPLICATIONS

The present disclosure claims priority to Chinese patent application No. 202410432660.0, titled “DEVIATION ACQUISITION METHOD AND DEVICE FOR SINGLE-PHOTON EMISSION COMPUTED TOMOGRAPHY SYSTEM”, filed on Apr. 10, 2024, the entire content of which is incorporated herein by reference.

### TECHNICAL FIELD

The present disclosure relates to the field of nuclear medicine technologies, and in particular, to a deviation acquisition method for a single-photon emission computed tomography system, a computer device, and a storage medium.

### BACKGROUND

Single-photon emission computed tomography (SPECT) technology, as a mature imaging technology in the field of nuclear medicine today, has been widely used in clinical testing. A SPECT system is provided with at least one system detector, and in order to obtain reliable detection results, it is necessary to accurately know the position, direction and variation of the system detector during the scanning process. However, due to the influence of various factors, the system detector often deviates from the expected position, affecting the reliability of the detection results.

In the related art, the deviation between the actual position and the expected position of the system detector can be estimated and corrected by measuring the radiation source. However, the accuracy of deviation of the SPECT system obtained in the above manner still needs to be improved.

### SUMMARY

In a first aspect, the present disclosure provides a deviation acquisition method for a single-photon emission computed tomography (SPECT) system. The method includes:
controlling a target object to move to a first target position through a translation stage, and determining an actual detection position of the target object detected by a system detector in a current pose;
obtaining at least one first expected pose deviation, the first expected pose deviation representing an estimated deviation between an expected pose and the current pose of the system detector;
determining at least one expected detection position of the target object based on the first target position and the at least one first expected pose deviation, and obtaining at least one first position difference between the at least one expected detection position and the actual detection position; and
determining an actual pose deviation between the expected pose and the current pose of the system detector based on the at least one first position difference corresponding to the at least one first expected pose deviation.

In an embodiment, determining the actual detection position of the target object detected by the system detector in the current pose includes:
determining a projection of the target object on a detection surface corresponding to the system detector when the target object is located at the first target position, the detection surface being a detection surface corresponding to the system detector in the current pose; and
determining the actual detection position of the target object detected by the system detector based on projection center of the projection on the detection surface.

In an embodiment, after determining the actual pose deviation between the expected pose and the current pose of the system detector based on the at least one first position difference corresponding to the at least one first expected pose deviation, the method further includes:
adjusting the current pose of the system detector, and re-determining the actual pose deviation between the expected pose and the current pose of the system detector after the pose adjustment to obtain the actual pose deviation of the system detector in each pose.

In an embodiment, after obtaining the actual pose deviation of the system detector in each pose, the method further includes:
controlling the target object to move to a second target position through the translation stage;
determining an actual position of the target object in a coordinate system of the SPECT system based on the actual position difference of the system detector in each pose and scan data acquired by the system detector for the target object;
obtaining at least one second expected pose deviation, the second expected pose deviation representing an estimated deviation between the coordinate system of the SPECT system and a coordinate system of the translation stage;
determining at least one predicted position of the target object in the coordinate system of the SPECT system based on the second target position and the at least one second expected pose deviation, and obtaining at least one second position difference between the at least one predicted position and the actual position; and
determining an actual pose deviation between the coordinate system of the SPECT system and the coordinate system of the translation stage based on the at least one second position difference corresponding to the at least one second expected pose deviation.

In an embodiment, determining the actual position of the target object in the coordinate system of the SPECT system based on the actual position difference of the system detector in each pose and the scan data acquired by the system detector for the target object includes:
obtaining the scan data acquired by the system detector for the target object;
correcting the scan data based on the actual pose deviation of the system detector in each pose, and performing image reconstruction based on the corrected scan data to obtain a reconstructed image; and
determining the actual position of the target object in the coordinate system of the SPECT system based on projection center of the target object on the reconstructed image.

In an embodiment, the actual detection position comprises actual detection positions respectively obtained when the target object is located at a plurality of first target positions, and the expected detection position comprises expected detection positions respectively obtained when the target object is located at a plurality of first target positions; and
obtaining the first position difference between the expected detection position and the actual detection position includes:
for each first target position, determining a distance between a corresponding expected detection position and the actual detection position;
determining the first position difference between the expected detection position and the actual detection position based on the distance corresponding to each first target position.

In an embodiment, the target object includes a radiation source disposed on a mechanical arm of the translation stage;
controlling the target object to move to the first target position through the translation stage includes:
sending a radiation source movement instruction to the translation stage, the radiation source movement instruction being configured to instruct the translation stage to control a movement of the mechanical arm based on displacement control information in the radiation source movement instruction, such that the radiation source moves to the corresponding first target position.

In an embodiment, determining the actual pose deviation between the expected pose and the current pose of the system detector based on the at least one first position difference corresponding to the at least one first expected pose deviation includes:
determining the first expected pose deviation corresponding to the smallest first position difference from the at least one first position difference as the actual pose deviation between the expected pose and the current pose of the system detector.

In an embodiment, after determining the projection of the target object on the detection surface corresponding to the system detector when the target object is located at the first target position, the method further includes:
performing Gaussian fitting on the projection in any two directions in the coordinate system of the SPECT system respectively to obtain peak values of the projection after the Gaussian fitting in the two directions;
determining projection center of the projection on the detection surface based on the peak values of the projection after the Gaussian fitting in the two directions.

In an embodiment, after performing image reconstruction based on the corrected scan data to obtain the reconstructed image, the method further includes:
performing Gaussian fitting on projection of the reconstructed image in three directions in the coordinate system of the SPECT system respectively to obtain peak values of the reconstructed image after the Gaussian fitting in the three directions; and
determining projection center of the target object on the reconstructed image based on the peak values of the reconstructed image after the Gaussian fitting in the three directions.

In a second aspect, the present disclosure further provides a deviation acquisition device for a single-photon emission computed tomography system. The device includes:
an actual detection position determination module configured to control a target object to move to a first target position through a translation stage, and determine an actual detection position of the target object detected by a system detector in a current pose;
a first deviation prediction module configured to obtain at least one first expected pose deviation, the first expected pose deviation representing an estimated deviation between an expected pose and the current pose of the system detector;
an expected detection position determination module configured to determine at least one expected detection position of the target object based on the first target position and the at least one first expected pose deviation, and obtain at least one first position difference between the at least one expected detection position and the actual detection position; and
a pose deviation determination module configured to determine an actual pose deviation between the expected pose and the current pose of the system detector based on the at least one first position difference corresponding to the at least one first expected pose deviation.

In a third aspect, the present disclosure further provides a computer device including a processor and a memory storing a computer program. The computer program, when executed by the processor, causes the processor to perform:
controlling a target object to move to a first target position through a translation stage, and determining an actual detection position of the target object detected by a system detector in a current pose;
obtaining at least one first expected pose deviation, the first expected pose deviation representing an estimated deviation between an expected pose and the current pose of the system detector;
determining at least one expected detection position of the target object based on the first target position and the at least one first expected pose deviation, and obtaining at least one first position difference between the at least one expected detection position and the actual detection position; and
determining an actual pose deviation between the expected pose and the current pose of the system detector based on the at least one first position difference corresponding to the at least one first expected pose deviation.

In a fourth aspect, the present disclosure further provides a non-transitory computer-readable storage medium storing a computer program. The computer program, when executed by a processor, causes the processor to perform:
controlling a target object to move to a first target position through a translation stage, and determining an actual detection position of the target object detected by a system detector in a current pose;
obtaining at least one first expected pose deviation, the first expected pose deviation representing an estimated deviation between an expected pose and the current pose of the system detector;
determining at least one expected detection position of the target object based on the first target position and the at least one first expected pose deviation, and obtaining at least one first position difference between the at least one expected detection position and the actual detection position; and
determining an actual pose deviation between the expected pose and the current pose of the system detector based on the at least one first position difference corresponding to the at least one first expected pose deviation.

In a fifth aspect, the present disclosure further provides a computer program product including a computer program. The computer program, when executed by a processor, causes the processor to perform:
controlling a target object to move to a first target position through a translation stage, and determining an actual detection position of the target object detected by a system detector in a current pose;
obtaining at least one first expected pose deviation, the first expected pose deviation representing an estimated deviation between an expected pose and the current pose of the system detector;
determining at least one expected detection position of the target object based on the first target position and the at least one first expected pose deviation, and obtaining at least one first position difference between the at least one expected detection position and the actual detection position; and
determining an actual pose deviation between the expected pose and the current pose of the system detector based on the at least one first position difference corresponding to the at least one first expected pose deviation.

One or more embodiments of the present disclosure will be described in detail below with reference to drawings. Other features, objects and advantages of the present disclosure will become more apparent from the description, drawings, and claims.

### BRIEF DESCRIPTION OF THE DRAWINGS

In order to describe the technical solutions of the embodiments of the present disclosure or the related art more clearly, the accompanying drawings required for describing the embodiments or for describing the related art will be briefly introduced as follows. Apparently, the accompanying drawings, in the following description, illustrate merely some embodiments of the present disclosure, for a person of ordinary skill in the art, other drawings can also be obtained based on these accompanying drawings without making any creative efforts.

FIG. 1 is a schematic flow chart of a deviation acquisition method for a single-photon emission computed tomography system in an embodiment.

FIG. 2 is a schematic diagram showing an actual pose and an expected pose of a system detector in an embodiment.

FIG. 3 is a schematic flow chart of a method for determining an actual pose deviation between a coordinate system of the SPECT system and a coordinate system of a translation stage in an embodiment.

FIG. 4 is a schematic diagram showing positions of the single-photon emission computed tomography system and a translation stage system in an embodiment.

FIG. 5 is a schematic flow chart of another deviation acquisition method for a single-photon emission computed tomography system in an embodiment.

FIG. 6 is a block diagram showing a configuration of a deviation acquisition device for a single-photon emission computed tomography system in an embodiment.

FIG. 7 is a diagram showing an internal configuration of a computer device in an embodiment.

FIG. 8 is a diagram showing an internal configuration of another computer device in an embodiment.

### DETAILED DESCRIPTION OF THE EMBODIMENTS

In order to make the objectives, technical solutions and advantages of the present disclosure more clearly understood, the present disclosure will be further described in detail with the accompanying drawings and embodiments. It should be understood that the specific embodiments described herein are only used to explain the present disclosure and not to limit the present disclosure.

In order to enable those skilled in the art to better understand the present disclosure, the related art is first introduced below.

Single-photon emission computed tomography (SPECT) technology, as a mature imaging technology in the field of nuclear medicine today, has been widely used in clinical testing. A single-photon emission computed tomography system (hereinafter referred to as a SPECT system) is provided with a detector. During the detection process, the detector performs different movements to complete the scanning of a patient, such as controlling the detector to rotate around the patient. In order to reconstruct the distribution and variations of a radioactive tracer in the patient's body from scan data, it is necessary to accurately determine positions, directions and corresponding variations of the detector during the scanning process. However, in a practical application, due to factors such as errors in rack installation, errors in motion control, or the weight of the detector, the detector often deviates from the expected position, resulting in a decrease in the quality of a reconstructed image and the appearance of artifacts in the reconstructed image, which seriously affects the quality of the detection result.

In the related art, the deviation between the actual position and the expected position of the detector can be estimated and corrected by measuring the radiation source. However, due to the influence of the geometric accuracy of the radiation source (such as the distance or direction error between the radiation sources), it is difficult to obtain an accurate deviation estimation result. In addition, the actual motion state of the detector can also be obtained through an external device (such as an optical capture method). However, due to problems such as the mechanical accuracy and installation error of the detector, there is still an error between the detector housing and the actual detection plane.

Based on this, the present disclosure provides a deviation acquisition method and device for a single-photon emission computed tomography system, a computer device, a storage medium and a computer program product, so as to at least solve the problem of low accuracy of deviation of the SPECT system obtained in the related art.

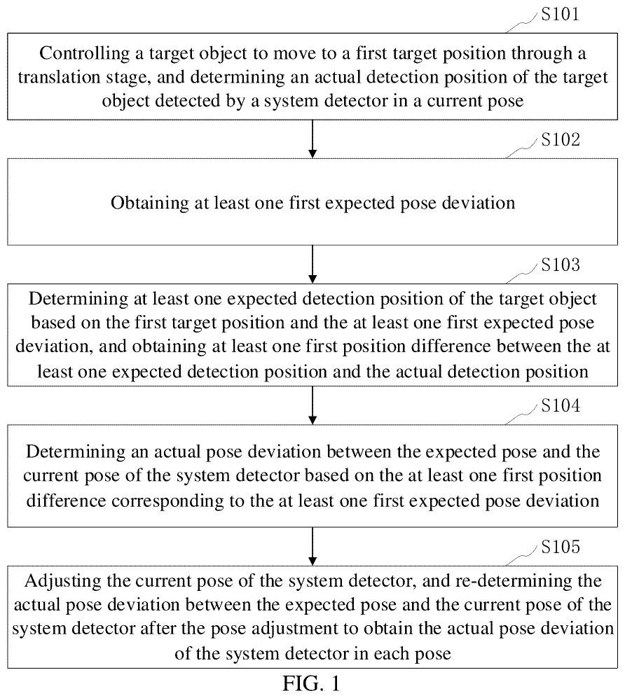

In an embodiment, as shown in FIG. 1, a deviation acquisition method for a single-photon emission computed tomography system is provided. The present embodiment is illustrated by applying the method to the SPECT system. It is understandable that the method can also be applied to a server, and can also be applied to a system including a terminal and a server, and can be implemented through an interaction between the terminal and the server. In this embodiment, the method includes the following steps S101 to S104.

In the step S101, a target object is controlled to move to a first target position through a translation stage, and determining an actual detection position of the target object detected by a system detector in a current pose.

The translation stage can be understood as a high-precision actuator. In some embodiments, the translation stage can be driven by a stepper motor therein to move in at least one axial direction among an x-axis direction, a y-axis direction and a z-axis direction, so as to translate the target object to a specified position, achieving high-precision micro-movement in any direction (for example, movement at the nanometer level or above). In some embodiments, the translation stage may be a patient bed.

Typically, the SPECT system includes at least one system detector. Each system detector includes a collimator and a detector. The pose data of the system detector may include a position and rotation angle of the detector, a position and rotation angle of the collimator, and a position and rotation angle of the collimator hole.

The target object may be a radiation source carrying a radioactive tracer.

In some embodiments, the translation stage can be configured to control the movement of the target object in at least one of the x-axis direction, the y-axis direction, or the z-axis direction. For example, in an embodiment, if the translation stage is a three-dimensional translation stage, the target object can be controlled to move arbitrarily in a three-dimensional space. If the translation stage is a two-dimensional translation stage, the target object can be controlled to move arbitrarily on a two-dimensional plane.

In the present embodiment, the target object can be controlled to move to a specified position through the translation stage. The specified position can be any position within a scan area of the SPECT system. To facilitate distinguishing it from other positions, this position is also referred to as the first target position. The first target position can be understood as a movement control result of the translation stage on the target object, i.e., the first target position can be obtained based on position control information input during the movement control process, and corresponds to a position of the target object in the real world, which can identify an actual position of the target object. By controlling the movement of the target object through the translation stage, it is possible to achieve precise control of the position of the target object and obtain accurate and reliable position information of the target object.

After the target object is controlled to move to the first target position through the translation stage, the system detector can be controlled to detect the target object located at the first target position in the current pose to identify the actual detection position of the target object.

The actual detection position can be understood as the position of the target object in the real world obtained by the system detector after detecting independently of the translation stage. In other words, the actual detection position can be an identification result obtained after the system detector identifies the position of the target object located at the first target position.

In the step S102, at least one first expected pose deviation is obtained. The first expected pose deviation represents an estimated deviation between an expected pose and the current pose of the system detector.

Taking the pose data of the system detector including the pose data of the detector as an example, the pose of the detector refers to the position and posture of the detector in the coordinate system of the SPECT system. The position of the detector may be a specific position of the detector in the three-dimensional space, such as the coordinates of the center point of the detector on the X, Y, and Z axes in the coordinate system of the SPECT system. The posture of the detector may be a rotation angle of the detector in the three-dimensional space, such as a rotation angle of the detector around a vertical axis (generally the Z axis), a rotation angle of the detector around a horizontal axis (generally the Y axis), or a rotation angle of the detector around its own axis (generally the X axis). The expected pose of the system detector may also be referred to as an ideal pose, which may be understood as the pose that the user desires the system detector to achieve. For example, a pose of the system detector input by the user in the SPECT system may be taken as the expected pose of the system detector. The current pose of the system detector may also be referred to as an actual pose of the system detector, i.e., the pose currently maintained by the system detector in the real world.

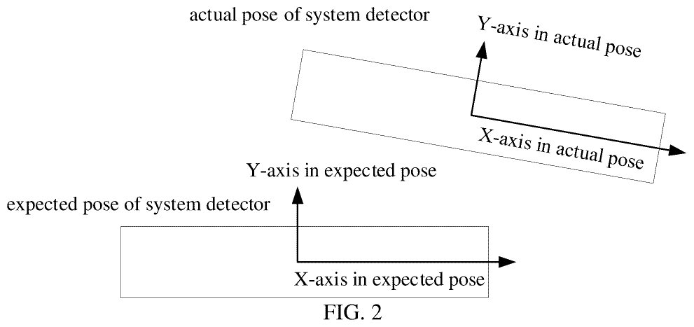

In a practical application, there may be a certain deviation between the expected pose and the actual pose of the system detector. FIG. 2 is a schematic diagram showing a pose deviation on a two-dimensional plane. For simplicity, FIG. 2 shows the pose deviation in the two-dimensional plane, while in an actual situation, the pose deviation is a deviation in the three-dimensional space. It can be seen from FIG. 2 that the expected pose of the system detector has a certain deviation in position and rotation angle relative to the actual pose.

In the present embodiment, the pose deviation between the expected pose and the current pose of the system detector can be predicted, i.e., the difference between the expected pose and the current pose of the system detector can be estimated in advance, and one or more expected pose deviations can be determined. The pose deviation can include a translation deviation between the expected pose and the current pose, and a deviation in the rotation angle between the expected pose and the current pose. For ease of distinction, the pose deviation can also be referred to as the first expected pose deviation, which can reflect the displacement and/or rotation angle of the expected pose relative to the current pose.

In the step S103, at least one expected detection position of the target object is determined based on the first target position and the at least one first expected pose deviation, and at least one first position difference between the at least one expected detection position and the actual detection position is obtained.

Specifically, after the target object is moved to the first target position, coordinate transformation may be performed based on the first target position to predict the position of the target object detected by the system detector when the system detector is in the expected pose. For example, if the first target position is a position determined by using the coordinate system of the translation stage as a reference system, the first target position can be transformed through a transformation relationship between the coordinate system of the translation stage and the coordinate system of the SPECT system, and a position transformation result can be taken as the position of the target object identified when the system detector is in the expected pose (i.e., ideal pose). Then, the position transformation result and the first expected pose deviation can be combined to predict the position of the target object detected by the system detector in consideration of the deviation between the expected pose and the current pose. This position is also referred to as the expected detection position or the predicted position. It can be understood that the expected detection position is the position obtained by theoretical calculation when the pose deviation is taken into account.

In the present embodiment, the position detected by the system detector in the expected pose can be predicted based on the first target position, and then combined with the first expected pose deviation to obtain the expected detection position corresponding to the target object when the system detector is in the current pose while considering the pose offset of the system detector.

It can be understood that the actual detection position acquired in advance is a position determined based on the detection data of the system detector, i.e., the actual detection position is the position actually detected by the system detector, and the expected detection position is a position calculated based on the first target position and the first expected pose deviation. The actual detection position and the expected detection position are the results obtained by identifying the same object (i.e., the position of the target object detected in the current pose of the system detector) in different ways. Ideally, the actual detection position is the same as the expected detection position. However, in a specific implementation, due to different estimates of the first expected pose deviation, there may be a difference between the actual detection position and the expected detection position. By comparing the difference between the actual detection position and the expected detection position, it can be determined whether the first expected pose deviation is accurate.

Based on this, after the expected detection position of the target object is determined, the position difference between the expected detection position and the actual detection position can be determined. For ease of distinction, the position difference is also referred to as the first position difference.

In the step S104, an actual pose deviation between the expected pose and the current pose of the system detector is determined based on the at least one first position difference corresponding to the at least one first expected pose deviation.

Specifically, when the at least one first expected pose deviation is obtained, for each first expected pose deviation, a corresponding first position difference under the first expected pose deviation can be obtained based on the step S103. It can be understood that the first position difference is related to the first expected pose deviation. The higher the accuracy of the first expected pose deviation, the smaller the first position difference. Based on this, the actual pose deviation between the expected pose and the current pose of the system detector can be determined based on the first position differences corresponding to the respective first expected pose deviations.

In an embodiment, if a plurality of first expected pose deviations Γ₁ are obtained in advance, after determining the first position differences F(Γ₁) corresponding to the first expected pose deviations Γ1, the first position differences F(Γ₁) can be compared to determine the corresponding first expected pose deviation Γ₁ when F(Γ₁) takes the minimum value, and the corresponding first expected pose deviation Γ₁ is determined as the actual pose deviation between the expected pose and the current pose of the system detector. For example, the actual pose deviation between the expected pose and the current pose can be determined based on the following formula:

$${Γ}^{†} = arg {min}_{Γ} F ( Γ )$$

where Γ^{†} is the actual pose deviation between the expected pose and the current pose, and Γ is the pose deviation, which may be the first expected pose deviation Γ₁ or the second expected pose deviation Γ₂.

In the above deviation acquisition method for a single-photon emission computed tomography system, the target object is first controlled to move to the first target position through the translation stage, and then the actual detection position of the target object detected by the system detector in the current pose can be determined, and the at least one first expected pose deviation can be obtained. The first expected pose deviation represents the estimated deviation between the expected pose and the current pose of the system detector. Further, the at least one expected detection position of the target object is determined based on the first target position and the at least one first expected pose deviation, and then the at least one first position difference between the at least one expected detection position and the actual detection position can be obtained. Further, the actual pose deviation between the expected pose and the current pose of the system detector is determined based on the at least one first position difference corresponding to the at least one first expected pose deviation. In this embodiment, since the position of the target object can be precisely controlled through the translation stage, the first target position accurately corresponds to the actual position of the target object. Therefore, the difference between the expected detection position, which is calculated based on the first target position and the first expected pose deviation, and the actual detection position can accurately represent the deviation between the expected pose and the current pose of the system detector, thereby effectively improving the accuracy of deviation estimation and correction.

In an embodiment, determining the actual detection position of the target object detected by the system detector in the current pose in the step S101 may include the following steps.

A projection of the target object on a detection surface corresponding to the system detector when the target object is located at the first target position is determined, and the actual detection position of the target object detected by the system detector is determined based on projection center of the projection on the detection surface.

The system detector may have a corresponding detection surface, and the detection surface may vary accordingly with the variation of the pose of the system detector. In this embodiment, the detection surface is a detection surface corresponding to the system detector in the current pose. Exemplarily, the detection surface corresponding to the system detector may be a flat surface, such as a flat detector, or the detection surface corresponding to the system detector may be a curved surface, such as a curved detector.

In a specific implementation, when the rays emitted by the target object hit the detection surface of the system detector, a corresponding projection will be formed on the detection surface. In this embodiment, when the target object moves to the first target position, the system detector in the current pose can be used to acquire the scan data of the target object, so as to obtain the projection of the target object on the detection surface of the system detector in the current pose.

After obtaining the projection of the target object on the detection surface, the projection center can be determined. In an embodiment, for the projection on the detection surface, Gaussian fitting can be performed on the projection in two directions perpendicular to each other in the coordinate system of the SPECT system. For example, the Gaussian fitting can be performed on the projection in the x-axis direction and the y-axis direction corresponding to the detection surface, respectively, and the projection center can be determined based on the peak values of the Gaussian fitting in the two directions.

Then, the actual detection position of the target object detected by the system detector can be determined based on the projection center. For example, a position of the projection center can be obtained, and the position of the projection center can be determined as the actual detection position of the target object.

In this embodiment, the detection surface corresponding to the system detector varies accordingly with the variation of the pose of the system detector. By obtaining the projection center of the target object on the detection surface, the actual detection position detected by the system detector in the current pose can be quickly and accurately obtained with the detection surface as the reference system.

In an embodiment, as shown in FIG. 1, after determining the actual pose deviation between the expected pose and the current pose of the system detector based on the at least one first position difference corresponding to the at least one first expected pose deviation in the step S104, the method may further include the following step S105.

In the step S105, the current pose of the system detector is adjusted, and the actual pose deviation between the expected pose and the current pose of the system detector after the pose adjustment is re-determined to obtain the actual pose deviation of the system detector in each pose.

In a specific implementation, after determining the actual pose deviation corresponding to the system detector in the current pose, the current pose of the system detector can be adjusted. For example, the SPECT system can adjust the pose of the system detector based on pose adjustment parameters and rotate the system detector to a new angle. In some embodiments, the pose adjustment of the system detector can be determined based on the pose used by the system detector in an actual detection process, i.e., the pose of the system detector can be adjusted to various poses used in the actual detection process.

After obtaining the new current pose, the actual pose deviation between the expected pose and the current pose of the system detector after pose adjustment can be re-determined based on the step S101 to step S104, thereby obtaining the actual pose deviation of the system detector in each pose.

In this embodiment, by adjusting the pose of the system detector and determining the actual pose deviation again, the actual pose deviation of the system detector in each pose can be obtained, and the data acquired by the system detector in different poses can be corrected.

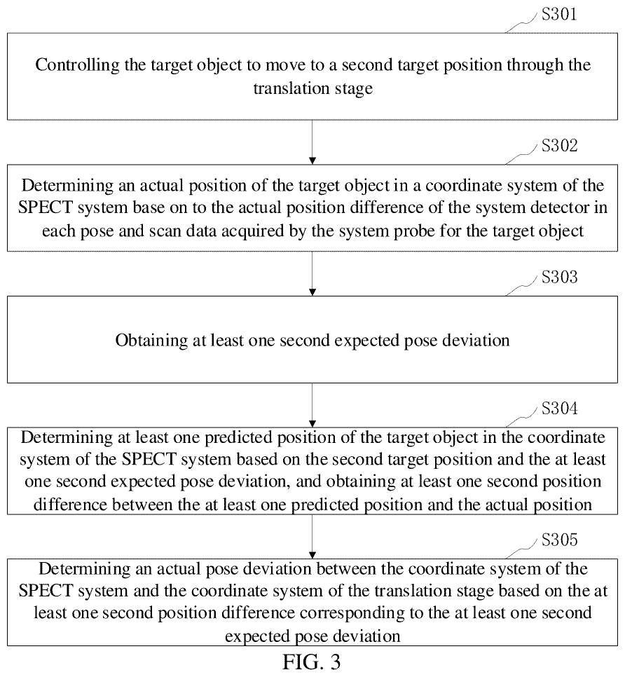

In an embodiment, as shown in FIG. 3, after obtaining the actual pose deviation of the system detector in each pose, the method may further include the following steps.

In the step S301, a target object is controlled to move to a second target position through the translation stage.

In a practical application, the target object can be controlled to move to a specified position through the translation stage. The specified position can be any position within the scan area of the SPECT system. For ease of distinction, the position can be referred to as the second target position. The second target position can be understood as a movement control result of the translation stage on the target object, i.e., the second target position can be obtained based on position control information input during the movement control process, and corresponds to a position of the target object in the real world, which can identify an actual position of the target object.

In the step S302, an actual position of the target object in a coordinate system of the SPECT system is determined based on the actual position difference of the system detector in each pose and scan data acquired by the system detector for the target object.

After the target object is moved to the second target position, the system detector can be used to acquire data on the target object in the corresponding pose to obtain the scan data. Then, the actual position of the target object in the coordinate system of the SPECT system can be determined based on the actual pose deviation of the system detector in each pose and the scan data.

In the step S303, at least one second expected pose deviation is obtained. The second expected pose deviation represents an estimated deviation between the coordinate system of the SPECT system and a coordinate system of the translation stage.

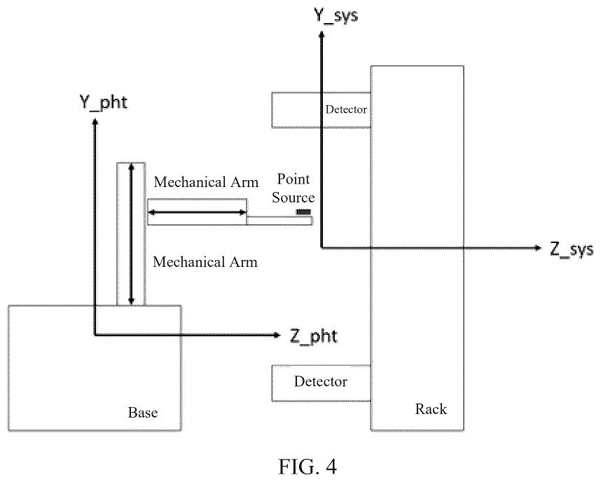

The coordinate system of the SPECT system is a coordinate system corresponding to the SPECT system, and the coordinate system of the translation stage is a coordinate system corresponding to the translation stage. There is a pose deviation between the coordinate system of the SPECT system (X-Y-Z_sys) and the coordinate system of the translation stage (X-Y-Z_pht). For example, FIG. 4 is a schematic diagram showing positions of the SPECT system and the translation stage. For simplicity, FIG. 4 shows the positions of the SPECT system and the translation stage on the two-dimensional plane, while in an actual situation, the positions of the SPECT system and the translation stage are positions in the three-dimensional space.

In the present embodiment, the pose deviation between the coordinate system of the SPECT system and the coordinate system of the translation stage can be predicted, i.e., the difference between the coordinate system of the SPECT system and the coordinate system of the translation stage can be estimated in advance, and one or more expected pose deviations can be determined. The pose deviation can include a translation deviation between the coordinate system of the SPECT system and the coordinate system of the translation stage, and a deviation in the rotation angle between the coordinate system of the SPECT system and the coordinate system of the translation stage. For ease of distinction, the pose deviation can also be referred to as the second expected pose deviation, which can reflect the displacement and/or rotation angle of the coordinate system of the SPECT system relative to the coordinate system of the translation stage.

In the step S304, at least one predicted position of the target object in the coordinate system of the SPECT system is determined based on the second target position and the at least one second expected pose deviation, and at least one second position difference between the at least one predicted position and the actual position is obtained.

Specifically, after the target object is moved to the second target position, the second target position can be a position determined with the coordinate system of the translation stage as the reference system, coordinate transformation may be performed on the second target position based on the second target position and the at least one second expected pose deviation to calculate the position of the target object in the coordinate system of the SPECT system, so as to obtain the at least one predicted position.

It can be understood that the actual position is a position determined based on the scan data acquired by the system detector, and the predicted position is a position calculated based on the second target position and the second expected pose deviation. The actual position and the predicted position are the results identify the same object (i.e., the position of the target object in the coordinate system of the SPECT system) in different ways. Ideally, the actual position is the same as the predicted position. However, in a specific implementation, due to different estimates of the second expected pose deviation, there may be a difference between the actual position and the predicted position. By comparing the difference between the actual position and the predicted position, it can be determined whether the second expected pose deviation is accurate.

Based on this, after the predicted position of the target object is determined, the position difference between the predicted position and the actual position can be determined. For ease of distinction, the position difference is also referred to as the second position difference.

In the step S305, an actual pose deviation between the coordinate system of the SPECT system and the coordinate system of the translation stage is determined based on the at least one second position difference corresponding to the at least one second expected pose deviation.

Specifically, for each second expected pose deviation of the at least one obtained second expected pose deviation, a corresponding second position difference under the second expected pose deviation can be obtained based on the step S304. It can be understood that the second position difference is related to the second expected pose deviation. The higher the accuracy of the second expected pose deviation, the smaller the second position difference. Based on this, the actual pose deviation between the coordinate system of the SPECT system and the coordinate system of the translation stage can be determined based on the second position differences corresponding to the respective second expected pose deviations.

In an embodiment, if a plurality of second expected pose deviations Γ₂ are obtained in advance, after determining the second position differences F(Γ₂) corresponding to the second expected pose deviations Γ₂, the second position differences F(Γ₂) can be compared to determine the corresponding second expected pose deviation Γ₂ when F(Γ₂) takes the minimum value, and the corresponding second expected pose deviation Γ₂ is determined as the actual pose deviation between the coordinate system of the SPECT system and the coordinate system of the translation stage.

In this embodiment, on the one hand, the actual position of the target object in the coordinate system of the SPECT system can be determined based on the actual pose deviation of the system detector in each pose, thereby improving the accuracy of the actual position detected by the system. On the other hand, since the position of the target object can be precisely controlled through the translation stage, the second target position accurately corresponds the actual position of the target object. Therefore, the difference between the predicted position, which is calculated based on the second target position and the second expected pose deviation, and the actual position can accurately represent the deviation between the coordinate system of the SPECT system and the coordinate system of the translation stage, thereby improving the accuracy of deviation estimation and correction.

In an embodiment, the step S302 of determining the actual position of the target object in the coordinate system of the SPECT system based on the actual position difference of the system detector in each pose and the scan data acquired by the system detector for the target object, may include the following steps.

The scan data acquired by the system detector for the target object is obtained; the scan data is corrected based on the actual pose deviation of the system detector in each pose, and image reconstruction is performed based on the corrected scan data to obtain a reconstructed image; and the actual position of the target object in the coordinate system of the SPECT system is determined based on projection center of the target object on the reconstructed image.

A method for correcting the scan data includes geometric correction and sensitivity correction. In some embodiments, the geometric correction refers to adjusting a geometric position reflected by projection data of the detector based on the actual pose deviation. For example, if the actual position of the detector in a certain pose (i.e., a certain scanning angle) is offset from the expected position, the geometric position reflected by the projection data of the detector can be corrected to the expected position through translation and rotation operations based on the actual pose deviation of the detector in this pose. In other embodiments, during the reconstruction process, the ideal position of the detector may be replaced by the actual position of the detector.

The sensitivity correction refers to adjusting a count rate of the projection data based on the actual pose deviation. For example, if the actual posture (e.g., rotation angle) of the detector at a certain scan angle is different from an expected posture, the count rate can be adjusted to compensate for the variation in sensitivity.

It can be understood that if the collimator or the collimator hole has a position difference relative to the detector, the above correction method is also applicable.

In a specific implementation, the system detector can obtain the scan data for the target object in a plurality of poses. Since there may be a difference between the actual pose and the expected pose of the system detector, after obtaining the scan data, the scan data can be corrected based on the actual pose deviation of the system detector in each pose to obtain the corrected scan data. The image reconstruction is performed based on the corrected scan data to obtain the reconstructed image corresponding to the target object.

After obtaining the reconstructed image, the projection center of the target object on the reconstructed image can be determined. In an embodiment, Gaussian fitting can be performed on the projection in three directions perpendicular to each other in the reconstructed image. The three directions perpendicular to each other can be the three coordinate axis directions in the coordinate system of the SPECT system. The Gaussian fitting can be performed on the projection in each direction, and the projection center is determined based on the peak values of the Gaussian fitting in the three directions.

Further, the actual position of the target object in the coordinate system of the SPECT system can be determined based on the projection center of the target object on the reconstructed image. For example, a position corresponding to the projection center may be obtained, and the position corresponding to the projection center may be determined as the actual position of the target object in the coordinate system of the SPECT system.

In this embodiment, the reconstructed image of the target object corresponds to the coordinate system of the SPECT system, and based on the projection center of the target object in the reconstructed image, the actual position of the target object in the coordinate system of the SPECT system can be quickly and accurately obtained.

In an embodiment, the actual detection position comprises actual detection positions respectively obtained when the target object is located at a plurality of first target positions, and the expected detection position comprises expected detection positions respectively obtained when the target object is located at a plurality of first target positions. In some embodiments, the target object includes only one point source, and the point source can be controlled to move to the plurality of different first target positions through the translation stage. For each first target position, the corresponding actual detection position and expected detection position when the target object is located at the first target position are obtained respectively. In some other embodiments, the target object may include multiple point sources. For the multiple point sources arranged at different positions, the actual detection positions and expected detection positions corresponding to the multiple point sources at their respective positions are respectively obtained.

Accordingly, the step S103 of obtaining the first position difference between the expected detection position and the actual detection position may include the following steps.

For each first target position, a distance between the corresponding expected detection position and the actual detection position is determined, and the first position difference between the expected detection position and the actual detection position is determined based on the distance corresponding to each first target position.

In a practical application, after obtaining the corresponding expected detection position and actual detection position when the target object is located at each first target position, the distance between the expected detection position and the actual detection position can be determined. Then, the distances at the respective first target position of the target object may be combined to determine the first position difference between the expected detection position and the actual detection position. For example, the first position difference can be determined based on the following formula:

$$F ( Γ ) = \frac{1}{2} {\sum }_{i = 1}^{m} {( {p}_{i} ( Γ ) - {c}_{i} )}^{2} = \frac{1}{2} {\sum }_{i = 1}^{m} {{f}_{i} ( Γ )}^{2}$$

$${f}_{i} ( Γ ) = {p}_{i} ( Γ ) - {c}_{i}$$

where F(Γ) represents the first position difference. When Γ takes the value of Γ₁, pᵢ(Γ₁) is the expected detection position corresponding to the i^{th} first target position under the first expected pose deviation Γ₁, cᵢ is the actual detection position corresponding to the i^{th} first target position, fᵢ(Γ₁) is the distance between the expected detection position and the actual detection position, and there are m first target positions in total.

In this embodiment, the first position difference can be determined by combining the distances between the expected detection positions and the actual detection positions when the target object is located at different first target positions, thereby reducing the deviation in the estimation process and improving the accuracy of estimation of the actual pose deviation between the expected pose and the current pose.

It can be understood that when determining the actual pose deviation between the coordinate system of the SPECT system and the coordinate system of the translation stage, the second position difference can also be determined by referring the above manner, i.e., the target object is moved to a plurality of different second target positions, and for each second target position, a distance between the corresponding predicted position and the actual position is determined, and the second position difference between the predicted position and the actual position is determined based on distances of the target object at the respective second target positions. The specific processing can refer to the above embodiment, which will not be repeated here.

In an embodiment, the target object includes a radiation source disposed on a mechanical arm of the translation stage. As shown in FIG. 4, the radiation source may be a point source, which may be fixed on the mechanical arm of the translation stage. Accordingly, the step S101 of controlling the target object to move to the first target position through the translation stage may include the following steps.

A radiation source movement instruction is sent to the translation stage. The radiation source movement instruction is configured to instruct the translation stage to control a movement of the mechanical arm based on displacement control information in the radiation source movement instruction, such that the radiation source moves to the corresponding first target position.

Specifically, the radiation source movement instruction may be sent to the translation stage to trigger the movement of the radiation source. After receiving the radiation source movement instruction, the translation stage can obtain the displacement control information in the radiation source movement instruction. The displacement control information can indicate a movement direction and a movement amplitude of the mechanical arm of the translation stage. Then, the translation stage can control the mechanical arm to move based on the displacement control information, such that the radiation source can move to the corresponding first target position.

In this embodiment, by disposing the radiation source on the mechanical arm, the position of the radiation source can be precisely controlled by controlling the displacement of the mechanical arm.

In order to enable those skilled in the art to better understand the technical solution of the present disclosure, the technical solution of the present disclosure is exemplarily illustrated below by combining an embodiment. It should be understood that the technical solution of the present disclosure is not limited to this embodiment.

In a specific implementation, the SPECT system and the translation stage have corresponding coordinate systems, respectively. X-Y-Z_sys represents the coordinate system of the SPECT system, and X-Y-Z_pht represents the coordinate system of the translation stage. The corresponding coordinate systems of the system detector are also different in the expected pose and the current pose. X-Y-Z_det_ide represents the coordinate system of the SPECT system detector in the expected pose, and X-Y-Z_det_rea represents the coordinate system of the SPECT system detector in the current pose.

X-Y-Z_sys and X-Y-Z_pht have the following transformation relationship:

$$\{ {X}_{pht} \} = \{ {R}_{sys , pht} \} \{ {X}_{sys} \} + \{ {T}_{sys , pht} \}$$

$${Γ}_{sys , pht} = \{ {R}_{sys , pht} , {T}_{sys , pht} \}$$

where {X_{sys}} and {Xₚₕₜ} are vectors with three rows and one column, {X_{sys}} represents coordinates of any point in the coordinate system X-Y-Z_sys of the system, {Xₚₕₜ} represents coordinates of any point in the coordinate system X-Y-Z_pht of the translation stage, {T_{sys,pht}} is a vector with three rows and one column, representing the translation amount between origins of the above two coordinate systems, {R_{sys,pht}} is a matrix with three rows and three columns, representing the rotation between the above two coordinate systems, and Γ_{sys,pht} is the parameter to be estimated.

In a certain pose (such as a certain rotation angle θ), X-Y-Z_det_ide and X-Y-Z_det_rea have the following transformation relationship:

$$\{ {X}_{det \_ ide} \} = \{ {R}_{ide , rea} \} \{ {X}_{det \_ rea} \} + \{ {T}_{ide , rea} \}$$

$${Γ}_{ide , rea} = \{ {R}_{ide , rea} , {T}_{ide , rea} \}$$

where {X_{det_rea}} and {X_{det_ide}} are vectors with three rows and one column, {X_{det_rea}} represents coordinates of any point in the coordinate system X-Y-Z_det_rea, {X_{det_ide}} represents coordinates of any point in the coordinate system X-Y-Z_det_ide, {T_{ide,rea}} is a vector with three rows and one column, representing the translation amount between origins of the above two coordinate systems, {R_{ide,rea}} is a matrix with three rows and three columns, representing the rotation between the above two coordinate systems, and Γ_{ide,rea} is the parameter to be estimated.

For ease of distinction in the following content, Γ_{ide,rea} is referred to as a first geometric parameter, and the first geometric parameter is configured to determine the actual pose deviation between the expected pose and the current pose of the system detector. Γ_{sys,pht} is referred to as a second geometric parameter, and the second geometric parameter is configured to determine the actual pose deviation between the coordinate system of the SPECT system and the coordinate system of the translation stage.

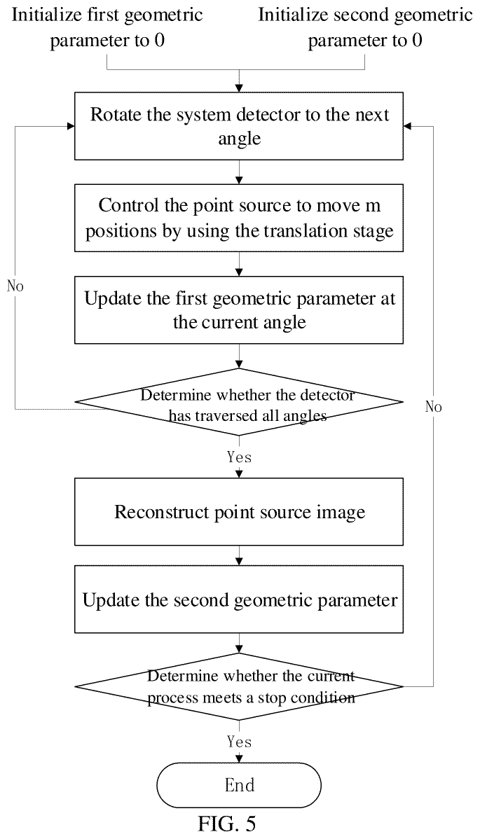

As shown in FIG. 5, the first geometric parameter and the second geometric parameter can be initialized to 0 first, and then the system detector can be rotated to a certain angle. The translation stage is triggered to control the radioactive point source to move to m positions (i.e., m first target positions), and data acquisition is performed, so as to determine the expected detection position and the actual detection position corresponding to the radioactive point source. It can be understood that when there is only one point source, the point source is needed to move to m positions and m data acquisitions are performed. When there are multiple point sources (for example, m point sources), data acquisition only needs to be performed once for the m point sources arranged at different positions. When the second geometric parameter remains unchanged, the expected detection positions and the actual detection positions are determined based on the point source at different positions, and the first geometric parameter at the current rotation angle is obtained.

After obtaining the first geometric parameter at the current rotation angle, it can be determined whether all rotation angles have been traversed. If all rotation angles have been not traversed, the current process returns to execute the step of rotating the system detector to the next rotation angle until the first geometric parameter at each angle is obtained.

Then, the translation stage can be triggered to control the radioactive point source to move to n positions (i.e., n second target positions), and the data acquisition is performed by using the system detector at the plurality of rotation angles to obtain the scan data. Further, a point source image is reconstructed based on the first geometric parameter and the scan data to determine the actual position of the point source in the coordinate system of the SPECT system, and the second geometric parameter is updated based on the corresponding predicted position. In some embodiments, a deviation threshold can be set to determine whether the second geometric parameter is less than or equal to the deviation threshold. If the second geometric parameter is less than or equal to the deviation threshold, it is determined that the current process meets a stop condition, i.e., the entire process can be ended. If the second geometric parameter is greater than the deviation threshold, it is determined that the current process does not meet the stop condition, and the current process needs to return to execute the step of rotating the system detector to the next rotation angle. Then, the second geometric parameter remains unchanged, and the first geometric parameter is updated again.

It should be understood that although the individual steps in the flow charts involved in the embodiments as described above are shown sequentially as indicated by arrows, the steps are not necessarily performed sequentially in the order indicated by the arrows. Unless explicitly stated herein, the execution of these steps is not strictly limited in any order and these steps can be performed in any other order. Moreover, at least some of the steps in the flow charts involved in the embodiments as described above may include multiple sub-steps or multiple stages that are not necessarily performed simultaneously, but may be performed at different moments. The order in which these sub-steps or stages are performed is not necessarily sequential, and these sub-steps or stages may be performed in turn or alternately with at least some of other steps or at least some of sub-steps or stages in other steps.

Based on the same inventive concept, embodiments of the present disclosure also provide a deviation acquisition device for a single-photon emission computed tomography system for implementing the deviation acquisition method for a single-photon emission computed tomography system as described above. The solution to the problem provided by the device is similar to the implementation of the method documented above, so the specific features in the one or more embodiments of the deviation acquisition device for a single-photon emission computed tomography system provided below may be understood with reference to the features of the deviation acquisition method for a single-photon emission computed tomography system above and will not be repeated here.

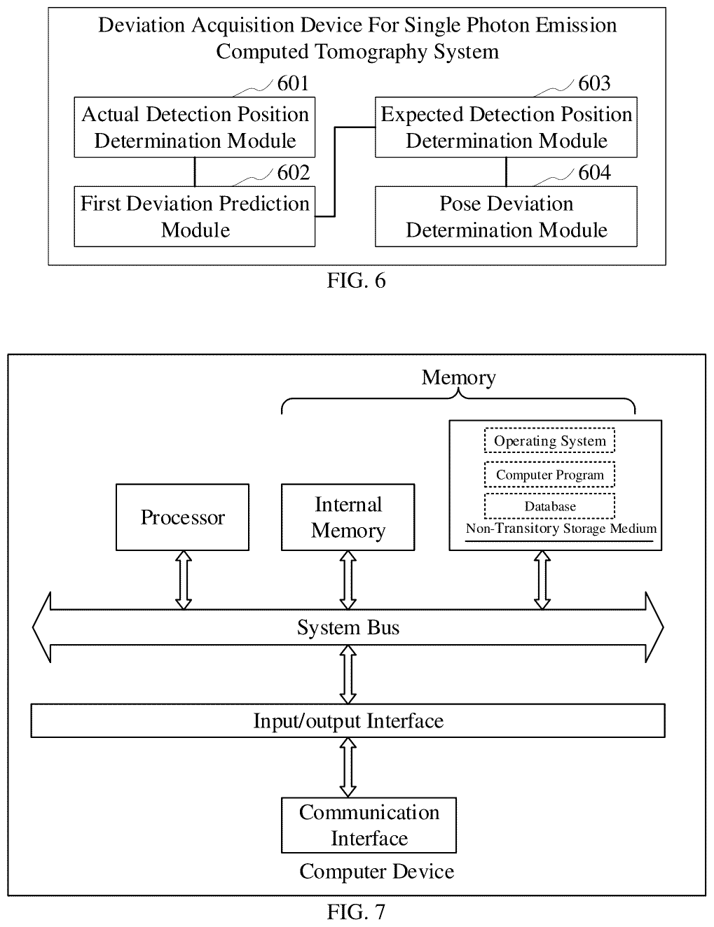

In an exemplary embodiment, as shown in FIG. 6, a deviation acquisition device for a single-photon emission computed tomography system is provided. The device includes the following modules.

An actual detection position determination module 601 is configured to control a target object to move to a first target position through a translation stage, and determine an actual detection position of the target object detected by a system detector in a current pose.

A first deviation prediction module 602 is configured to obtain at least one first expected pose deviation, the first expected pose deviation represents an estimated deviation between an expected pose and the current pose of the system detector.

An expected detection position determination module 603 is configured to determine at least one expected detection position of the target object based on the first target position and the at least one first expected pose deviation, and obtain at least one first position difference between the at least one expected detection position and the actual detection position.

A pose deviation determination module 604 is configured to determine an actual pose deviation between the expected pose and the current pose of the system detector based on the at least one first position difference corresponding to the at least one first expected pose deviation.

In an embodiment, the actual detection position determination module 601 is configured to determine a projection of the target object on a detection surface corresponding to the system detector when the target object is located at the first target position, the detection surface being a detection surface corresponding to the system detector in the current pose; and determine the actual detection position of the target object detected by the system detector based on projection center of the projection on the detection surface.

In an embodiment, the device is further configured to adjust the current pose of the system detector, and re-determine the actual pose deviation between the expected pose and the current pose of the system detector after the pose adjustment to obtain the actual pose deviation of the system detector in each pose.

In an embodiment, the device further includes the following modules.

A second target position moving module is configured to control the target object to move to a second target position through the translation stage.

An actual position determination module is configured to determine an actual position of the target object in a coordinate system of the SPECT system based on the actual position difference of the system detector in each pose and scan data acquired by the system detector for the target object.

A second deviation prediction module is configured to obtain at least one second expected pose deviation. The second expected pose deviation represents an estimated deviation between the coordinate system of the SPECT system and a coordinate system of the translation stage.

A predicted position determination module is configured to determine at least one predicted position of the target object in the coordinate system of the SPECT system based on the second target position and the at least one second expected pose deviation, and obtain at least one second position difference between the at least one predicted position and the actual position.

A coordinate system pose deviation determination module is configured to determine an actual pose deviation between the coordinate system of the SPECT system and the coordinate system of the translation stage based on the at least one second position difference corresponding to the at least one second expected pose deviation.

In an embodiment, the actual position determination module is configured to obtain the scan data acquired by the system detector for the target object; correct the scan data based on the actual pose deviation of the system detector in each pose, and perform image reconstruction based on the corrected scan data to obtain a reconstructed image; and determine the actual position of the target object in the coordinate system of the SPECT system based on projection center of the target object on the reconstructed image.

In an embodiment, the actual detection position comprises actual detection positions respectively obtained when the target object is located at a plurality of first target positions, and the expected detection position comprises expected detection positions respectively obtained when the target object is located at a plurality of first target positions. The expected detection position determination module 603 is configured to for each first target position, determine a distance between a corresponding expected detection position and the actual detection position; determine the first position difference between the expected detection position and the actual detection position based on the distance corresponding to each first target position.

In an embodiment, the target object includes a radiation source disposed on a mechanical arm of the translation stage. The actual detection position determination module 601 is configured to send a radiation source movement instruction to the translation stage. The radiation source movement instruction is configured to instruct the translation stage to control a movement of the mechanical arm based on displacement control information in the radiation source movement instruction, such that the radiation source moves to the corresponding first target position.

In an embodiment, the pose deviation determination module 604 is configured to compare the at least one first position difference, and determine the first expected pose deviation corresponding to the first position difference with a smallest value as the actual pose deviation between the expected pose and the current pose of the system detector.

In an embodiment, the actual detection position determination module 601 is further configured to perform Gaussian fitting on the projection in any two directions in the coordinate system of the SPECT system respectively to obtain peak values of the projection after the Gaussian fitting in the two directions; and determine projection center of the projection on the detection surface based on the peak values of the projection after the Gaussian fitting in the two directions.

In an embodiment, the expected detection position determination module 603 is further configured to perform Gaussian fitting on projection of the reconstructed image in three directions in the coordinate system of the SPECT system respectively to obtain peak values of the reconstructed image after the Gaussian fitting in the three directions; and determine projection center of the target object on the reconstructed image based on the peak values of the reconstructed image after the Gaussian fitting in the three directions.

The individual modules in the above deviation acquisition device for a single-photon emission computed tomography system can be implemented in whole or in part by software, hardware and combinations thereof. Each of the above modules may be embedded in hardware form or independent of a processor in a computer device, or may be stored in software form on a memory in the computer device so that the processor can be called to perform the operations corresponding to each of the above modules.

In an exemplary embodiment, a computer device is provided, which may be a server. A diagram illustrating an internal configuration of the computer device may be shown in FIG. 7. The computer device includes a processor, a memory, an input/output (I/O) interface, and a communication interface. The processor, the memory and the input/output interface are connected via a system bus, and the communication interface is connected to the system bus via the input/output interface. The processor of the computer device is configured to provide computing and control capabilities. The memory of the computer device includes a non-transitory storage medium and an internal memory. The non-transitory storage medium stores operating systems, computer programs and databases. The internal memory provides an environment for the operation of the operating systems and the computer programs in the non-transitory storage medium. The database of the computer device is configured to store the pose data. The input/output interface of the computer device is configured to exchange information between the processor and external devices. The communication interface of the computer device is configured to communicate with external terminals via a network connection. When the computer program is executed by the processor, a deviation acquisition method for a single-photon emission computed tomography imaging system is implemented.

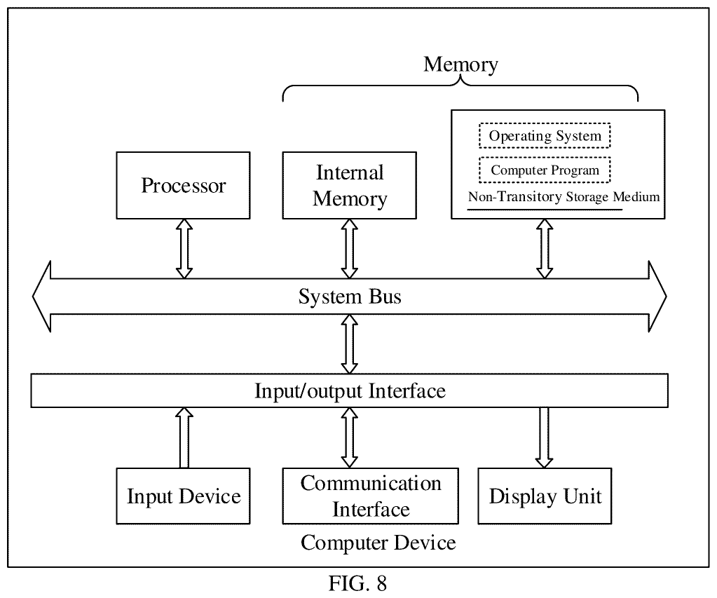

In an exemplary embodiment, a computer device is provided, which may be a terminal. A diagram illustrating an internal configuration of the computer device may be shown in FIG. 8. The computer device includes a processor, a memory, an input/output interface, a communication interface, a display unit, and an input device. The processor, the memory and the input/output interface are connected via a system bus, and the communication interface, the display unit and the input device are connected to the system bus via the input/output interface. The processor of the computer device is configured to provide computing and control capabilities. The memory of the computer device includes a non-transitory storage medium and an internal memory. The non-transitory storage medium stores operating systems and computer programs. The internal memory provides an environment for the operation of the operating systems and the computer programs in the non-transitory storage medium. The input/output interface of the computer device is configured to exchange information between the processor and external devices. The communication interface of the computer device is configured to communicate with external terminals in wired or wireless mode, which can be realized by WIFI, mobile cellular network, near field communication (NFC) or other technologies. The computer programs are executed by the processor in order to implement a deviation acquisition method for a single-photon emission computed tomography system. The display unit of the computer device is configured to form a visually visible picture, which may be a display screen, a projection device or a virtual reality imaging device. The display screen may be an LCD or e-ink display, and the input device of the computer device may be a touch layer covered by the display screen, or a key, trackball or trackpad set on the housing of the computer device, or an external keyboard, trackpad or mouse, etc.

It should be understood by a person of ordinary skill in the art that the configuration illustrated in FIGS. 7 and 8 is only a block diagram of part of the configuration related to the solution of the present disclosure, and does not constitute a limitation on the computer device to which the solution of the present disclosure is applied. A specific the computer device may include more or less components than those shown in the figure, or may combine some components, or may have a different arrangement of components.

In an embodiment, a computer device is provided, including a processor and a memory storing a computer program. When the processor executes the computer program the steps in the above method embodiments are implemented.

In an embodiment, a non-transitory computer-readable storage medium is provided, having a computer program stored thereon. When the computer program is executed by a processor, the steps in the above method embodiments are implemented.

In an embodiment, a computer program product is provided, including a computer program. When the computer program is executed by a processor, the steps in the above method embodiments are implemented.

It should be noted that the user information (including but not limited to user device information, user personal information, etc.) and data (including but not limited to data used for analysis, stored data, displayed data, etc.) involved in the present disclosure are information and data authorized by the user or fully authorized by all parties, and the collection, use and processing of relevant data need to comply with relevant regulations.

A person of ordinary skill in the art may understand that implementation of all or part of the processes in the methods of the above embodiments may be completed by instructing the relevant hardware through a computer program. The computer program may be stored in a non-transitory computer-readable storage medium. When the computer program is executed, it may include the processes of the respective methods based on the foregoing embodiments. Any reference to memory, database or other medium used of the embodiments provided in the present disclosure may include at least one of a non-transitory or a transitory memory. The non-transitory memory may include a read-only memory (ROM), a magnetic tape, a floppy disk, a flash memory, an optical memory, a high-density embedded non-transitory memory, a resistive random-access memory (ReRAM), a magneto resistive random-access memory (MRAM), a ferroelectric random-access memory (FRAM), a phase change memory (PCM), or a graphene memory, etc. The transitory memory may include a random-access memory (RAM) or an external cache memory, etc. As an illustration rather than a limitation, the random-access memory may be in various forms, such as a static random-access memory (SRAM) or a dynamic random-access memory (DRAM), etc. The databases involved in the embodiments provided by the present disclosure may include at least one of a relational database and a non-relational database. The non-relational database may include, but is not limited to, a blockchain-based distributed database, etc. The processor involved in the embodiments provided by the present disclosure may be, but is not limited to, a general purpose processor, a central processor, a graphics processor, a digital signal processor, a programmable logic device, a data processing logic device based on quantum computation, and the like.

The technical features in the above embodiments may be combined arbitrarily. For concise description, not all possible combinations of the technical features in the above embodiments are described. However, provided that they do not conflict with each other, all combinations of the technical features are to be considered to be within the scope of protection of the present disclosure.

The above-mentioned embodiments only describe several implementations of the present disclosure, and their description is specific and detailed, but should not be understood as a limitation on the protection scope of the present disclosure. It should be noted that, for a person of ordinary skill in the art, various variations and improvements can be further made without departing from the conception of the present disclosure, and these all fall within the protection scope of the present disclosure. Therefore, the protection scope of the present disclosure should be subject to the appended claims.

What is claimed is:

1. A deviation acquisition method for a single-photon emission computed tomography (SPECT) system, comprising:
controlling a target object to move to a first target position through a translation stage, and determining an actual detection position of the target object detected by a system detector in a current pose;
obtaining at least one first expected pose deviation, the first expected pose deviation representing an estimated deviation between an expected pose and the current pose of the system detector;
determining at least one expected detection position of the target object based on the first target position and the at least one first expected pose deviation, and obtaining at least one first position difference between the at least one expected detection position and the actual detection position; and
determining an actual pose deviation between the expected pose and the current pose of the system detector based on the at least one first position difference corresponding to the at least one first expected pose deviation.

2. The deviation acquisition method of claim 1, wherein determining the actual detection position of the target object detected by the system detector in the current pose comprises:
determining a projection of the target object on a detection surface corresponding to the system detector when the target object is located at the first target position, the detection surface being a detection surface corresponding to the system detector in the current pose; and
determining the actual detection position of the target object detected by the system detector based on projection center of the projection on the detection surface.

3. The deviation acquisition method of claim 1, wherein after determining the actual pose deviation between the expected pose and the current pose of the system detector based on the at least one first position difference corresponding to the at least one first expected pose deviation, the method further comprises:
adjusting the current pose of the system detector, and re-determining the actual pose deviation between the expected pose and the current pose of the system detector after the pose adjustment to obtain the actual pose deviation of the system detector in each pose.

4. The deviation acquisition method of claim 3, wherein after obtaining the actual pose deviation of the system detector in each pose, the method further comprises:
controlling the target object to move to a second target position through the translation stage;
determining an actual position of the target object in a coordinate system of the SPECT system based on the actual position difference of the system detector in each pose and scan data acquired by the system detector for the target object;
obtaining at least one second expected pose deviation, the second expected pose deviation representing an estimated deviation between the coordinate system of the SPECT system and a coordinate system of the translation stage;
determining at least one predicted position of the target object in the coordinate system of the SPECT system based on the second target position and the at least one second expected pose deviation, and obtaining at least one second position difference between the at least one predicted position and the actual position; and
determining an actual pose deviation between the coordinate system of the SPECT system and the coordinate system of the translation stage based on the at least one second position difference corresponding to the at least one second expected pose deviation.

5. The deviation acquisition method of claim 4, wherein determining the actual position of the target object in the coordinate system of the SPECT system based on the actual position difference of the system detector in each pose and the scan data acquired by the system detector for the target object comprises:
obtaining the scan data acquired by the system detector for the target object;
correcting the scan data based on the actual pose deviation of the system detector in each pose, and performing image reconstruction based on the corrected scan data to obtain a reconstructed image; and
determining the actual position of the target object in the coordinate system of the SPECT system based on projection center of the target object on the reconstructed image.

6. The deviation acquisition method of claim 1, wherein the actual detection position comprises actual detection positions respectively obtained when the target object is located at a plurality of first target positions, and the expected detection position comprises expected detection positions respectively obtained when the target object is located at a plurality of first target positions; and
obtaining the first position difference between the expected detection position and the actual detection position comprises:
for each first target position, determining a distance between a corresponding expected detection position and the actual detection position; and
determining the first position difference between the expected detection position and the actual detection position based on the distance corresponding to each first target position.

7. The deviation acquisition method of claim 1, wherein the target object comprises a radiation source disposed on a mechanical arm of the translation stage; and
wherein controlling the target object to move to the first target position through the translation stage comprises:
sending a radiation source movement instruction to the translation stage, the radiation source movement instruction being configured to instruct the translation stage to control a movement of the mechanical arm based on displacement control information in the radiation source movement instruction, such that the radiation source moves to the corresponding first target position.

8. The deviation acquisition method of claim 1, wherein determining the actual pose deviation between the expected pose and the current pose of the system detector based on the at least one first position difference corresponding to the at least one first expected pose deviation comprises:
determining the first expected pose deviation corresponding to the smallest first position difference from the at least one first position difference as the actual pose deviation between the expected pose and the current pose of the system detector.

9. The deviation acquisition method of claim 2, wherein after determining the projection of the target object on the detection surface corresponding to the system detector when the target object is located at the first target position, the method further comprises:
performing Gaussian fitting on the projection in any two directions in the coordinate system of the SPECT system respectively to obtain peak values of the projection after the Gaussian fitting in the two directions; and
determining projection center of the projection on the detection surface based on the peak values of the projection after the Gaussian fitting in the two directions.

10. The deviation acquisition method of claim 5, wherein after performing image reconstruction based on the corrected scan data to obtain the reconstructed image, the method further comprises:
performing Gaussian fitting on projection of the reconstructed image in three directions in the coordinate system of the SPECT system respectively to obtain peak values of the reconstructed image after the Gaussian fitting in the three directions; and
determining a projection center of the target object on the reconstructed image based on the peak values of the reconstructed image after the Gaussian fitting in the three directions.

11. A computer device comprising a processor and a memory storing a computer program, wherein the computer program, when executed by the processor, causes the processor to perform:
controlling a target object to move to a first target position through a translation stage, and determining an actual detection position of the target object detected by a system detector in a current pose;
obtaining at least one first expected pose deviation, the first expected pose deviation representing an estimated deviation between an expected pose and the current pose of the system detector;
determining at least one expected detection position of the target object based on the first target position and the at least one first expected pose deviation, and obtaining at least one first position difference between the at least one expected detection position and the actual detection position; and
determining an actual pose deviation between the expected pose and the current pose of the system detector based on the at least one first position difference corresponding to the at least one first expected pose deviation.

12. The computer device of claim 11, wherein determining the actual detection position of the target object detected by the system detector in the current pose comprises:
determining a projection of the target object on a detection surface corresponding to the system detector when the target object is located at the first target position, the detection surface being a detection surface corresponding to the system detector in the current pose; and
determining the actual detection position of the target object detected by the system detector based on projection center of the projection on the detection surface.

13. The computer device of claim 11, wherein after determining the actual pose deviation between the expected pose and the current pose of the system detector based on the at least one first position difference corresponding to the at least one first expected pose deviation, the computer device further comprises:
adjusting the current pose of the system detector, and re-determining the actual pose deviation between the expected pose and the current pose of the system detector after the pose adjustment to obtain the actual pose deviation of the system detector in each pose.

14. The computer device of claim 13, wherein after obtaining the actual pose deviation of the system detector in each pose, the computer device further comprises:
controlling the target object to move to a second target position through the translation stage;
determining an actual position of the target object in a coordinate system of the SPECT system based on the actual position difference of the system detector in each pose and scan data acquired by the system detector for the target object;
obtaining at least one second expected pose deviation, the second expected pose deviation representing an estimated deviation between the coordinate system of the SPECT system and a coordinate system of the translation stage;
determining at least one predicted position of the target object in the coordinate system of the SPECT system based on the second target position and the at least one second expected pose deviation, and obtaining at least one second position difference between the at least one predicted position and the actual position; and
determining an actual pose deviation between the coordinate system of the SPECT system and the coordinate system of the translation stage based on the at least one second position difference corresponding to the at least one second expected pose deviation.

15. The computer device of claim 14, wherein determining the actual position of the target object in the coordinate system of the SPECT system based on the actual position difference of the system detector in each pose and the scan data acquired by the system detector for the target object comprises:
obtaining the scan data acquired by the system detector for the target object;
correcting the scan data based on the actual pose deviation of the system detector in each pose, and performing image reconstruction based on the corrected scan data to obtain a reconstructed image; and
determining the actual position of the target object in the coordinate system of the SPECT system based on projection center of the target object on the reconstructed image.

16. The computer device of claim 11, wherein the actual detection position comprises actual detection positions respectively obtained when the target object is located at a plurality of first target positions, and the expected detection position comprises expected detection positions respectively obtained when the target object is located at a plurality of first target positions;
obtaining the first position difference between the expected detection position and the actual detection position comprises:
for each first target position, determining a distance between a corresponding expected detection position and the actual detection position; and
determining the first position difference between the expected detection position and the actual detection position based on the distance corresponding to each first target position.

17. The computer device of claim 11, wherein the target object comprises a radiation source disposed on a mechanical arm of the translation stage; and
wherein controlling the target object to move to the first target position through the translation stage comprises:
sending a radiation source movement instruction to the translation stage, the radiation source movement instruction being configured to instruct the translation stage to control a movement of the mechanical arm based on displacement control information in the radiation source movement instruction, such that the radiation source moves to the corresponding first target position.

18. The computer device of claim 11, wherein determining the actual pose deviation between the expected pose and the current pose of the system detector based on the at least one first position difference corresponding to the at least one first expected pose deviation comprises:
determining the first expected pose deviation corresponding to the smallest first position difference from the at least one first position difference as the actual pose deviation between the expected pose and the current pose of the system detector.

19. The computer device of claim 12, wherein after determining the projection of the target object on the detection surface corresponding to the system detector when the target object is located at the first target position, the computer device further comprises:
performing Gaussian fitting on the projection in any two directions in the coordinate system of the SPECT system respectively to obtain peak values of the projection after the Gaussian fitting in the two directions;
determining projection center of the projection on the detection surface based on the peak values of the projection after the Gaussian fitting in the two directions.

20. A non-transitory computer-readable storage medium storing a computer program, wherein the computer program, when executed by a processor, causes the processor to perform:
controlling a target object to move to a first target position through a translation stage, and determining an actual detection position of the target object detected by a system detector in a current pose;
obtaining at least one first expected pose deviation, the first expected pose deviation representing an estimated deviation between an expected pose and the current pose of the system detector;
determining at least one expected detection position of the target object based on the first target position and the at least one first expected pose deviation, and obtaining at least one first position difference between the at least one expected detection position and the actual detection position; and
determining an actual pose deviation between the expected pose and the current pose of the system detector based on the at least one first position difference corresponding to the at least one first expected pose deviation.
